(19) United States
(12) Patent Application Publication
(10) Pub. No.: US 2018/0277256 A1
(43) Pub. Date: Sep. 27, 2018

(54) METHOD, APPARATUS AND SOFTWARE FOR IDENTIFYING RESPONDERS IN A CLINICAL ENVIRONMENT

(71) Applicant: Acorda Therapeutics, Inc., Ardsley, NY (US)

(72) Inventors: Ron Cohen, Irvington, NY (US); Andrew R. Blight, Mahopac, NY (US); Lawrence Marinucci, White Plains, NY (US)

(21) Appl. No.: 15/996,338

(22) Filed: Jun. 1, 2018

Related U.S. Application Data
(63) Continuation of application No. 11/659,456, filed on Dec. 10, 2008, now abandoned.
(60) Provisional application No. 60/719,896, filed on Sep. 23, 2005.

(51) Int. Cl. G16H 50/70; G16H 10/60; G16H 10/20

(52) U.S. Cl. CPC G16H 50/70; G16H 10/20; G16H 10/60

(57) ABSTRACT

A process for determining responders in clinical testing environments that involves, inter alia, detecting treatment response through the use of small numbers of measurements of randomly varying outcome variables in individual clinical trial subjects, and by analyzing the measurements in such a way as to eliminate troublesome variables, such as a spontaneous population variability.

### BACKGROUND

Clinical researchers have the ongoing problem of not being able to accurately predict or plan future trials, and are not able to salvage or otherwise learn from failed clinical trials. There are many reasons for this. First, functional measurements from clinical trial subjects with certain kinds of conditions like MS and other ailments can vary extensively and randomly over time. This is problematic, because if these measurements are to be used as treatment outcome measures, the spontaneous variability can obscure the treatment-related effects. This interference between spontaneous and induced changes may be particularly problematic under conditions where only a subset of trial subjects respond to treatment. Under these conditions, the treatment effect in the responsive subjects may be diluted by the non-responders in addition to the contamination of spontaneous variability.

Moreover, clinical trials also frequently rely on just a few, intermittent measurements at widely spaced clinic visits. These few sample measurements will not adequately represent the full range of variation of the outcome variable, either during the baseline comparison period or during the treatment period. Where the magnitude of the spontaneous variability of the population is large compared to the expected treatment effect in the individual, it can be difficult to determine the presence of response to treatment based on the average difference between baseline and treatment periods. A single large outlying value in one direction or the other from the mean may mask a smaller but consistent response or alternatively produce the impression of a response that is not actually consistent during the treatment period. Thus, there is a need for detecting a consistent response to treatment over time without encountering false results from the above-mentioned variables.

It is therefore an object of the invention to provide a means to detect true, consistent response to treatment over time using small numbers of measurements from on-treatment and off-treatment periods has been devised to provide a solution for this problem.

It is further an object of the present invention to provide a method that involves examining the frequency with which values measured during the on-treatment period lie outside the range of values recorded during the off-treatment period(s) of the trial.

### SUMMARY OF THE INVENTION

This invention relates to a method, apparatus, and computer software application that can be used to analyze therapeutic effect of a treatment of patients in a clinical environment.

More specifically, the present invention may be utilized to analyze the response of patients in a clinical environment for many different types of afflictions, including, but not limited to, neurological disorders such as multiple sclerosis, spinal cord injuries, Alzheimer's disease and ALS.

One embodiment of the present invention relates to a method, apparatus and software program for analyzing clinical patient treatment data in order to predict future clinical trials.

Another embodiment of the present invention relates to a method, apparatus and software program for analyzing clinical patient treatment data in order to derive value from completed clinical trials, regardless of the outcome of the particular trial.

Another embodiment of the present invention relates to a method, apparatus and software program fur selecting individuals based on responsiveness to a treatment. The method comprises identifying a plurality of individuals; administering a test to each individual prior to a treatment period; administering a treatment to one or more of the individuals during the treatment period; administering the test a plurality of times to each individual during the treatment period; and selecting one or more individuals, wherein the selected individuals exhibit an improved performance during a majority of the tests administered during the treatment period as compared to the test administered prior to the treatment period. In certain embodiments, the method may further comprise administering, the test to each individual after the treatment period, wherein the selected individuals further exhibit an improved performance during a majority of the tests administered during the treatment period as compared to the test administered after the treatment period.

A farther embodiment relates to a method of selecting individuals based on responsiveness to a treatment, the method comprising identifying a plurality of individuals; administering a test to each individual prior to a treatment period; administering a treatment to one or more of the individuals during the treatment period; administering the test a plurality of times to each individual during the treatment period; administering the test to each individual alter the treatment period; and selecting one or more individuals, wherein the selected individuals exhibit an improved performance during a majority of the tests administered during the treatment period as compared to the better performance of the test administered prior to the treatment period and the test administered after the treatment period.

### BRIEF DESCRIPTION OF THE DRAWINGS

FIG. 1 is an exemplary flow diagram showing one way in which the inventive process may be put forth in a computer aided embodiment so treatment data from a clinical trial of a given number of patients may analyzed to determine the responders therein;

FIG. 2 is an exemplary flow diagram showing one way in which the probability distribution generation of the inventive process may be put forth in a computer aided embodiment in order to offer a comparative baseline against responder values;

FIG. 3 is a generalized system level block diagram of an exemplary system employing the inventive process described herein; and

FIGS. 4 (a)-4 (d) are histograms and distribution graphs of responder and non-responder populations shown in the context of the an illustrative utilization of the present invention.

### DETAILED DESCRIPTION OF THE INVENTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

The terms used herein have meanings recognized and known to those of skill in the art, however, for convenience and completeness, particular terms and their meanings are set forth below.

must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

“Software” means all forms of electronically executable code, regardless of the language employed for coding, specific system architecture coded for, and regardless of storage medium utilized (disk, download, ASP, etc.).

The terms “patient” and “subject” mean all animals including humans. Examples of patients or subjects include humans, cows, dogs, cats, goats, sheep, rats, pigs, etc.

One aspect of the invention therefore relates to a process of providing tier the above mentioned frequency to be compared between treatment and control groups, as well as with the predictions of a simple computer model based on random number generation. Hence, it there are j measurements made during treatment and k measurements made during the non-treatment period, a computer model can be generated that will predict the frequency with which a given subset of the measurements will exceed the largest of the k off treatment measurements. This is effectuated by using the method and the computer program of the present invention to generate many thousands of strings of j+k random numbers within a preset range and testing the frequency with which numbers in the j set exceed all numbers in the k set. Over the course of many thousand iterations, it will be possible to determine the probability that 1, 2, 3 . . . j of the j set will exceed all the k set within any one iteration.

By way of just one illustration, when j and k are small integers (say >3, <8) there will be a relatively high probability that just (or at least) one of the j set exceeds the maximum of the k set, but the probability wilt decrease rapidly for higher numbers of the j set, with the least probability that all of the numbers in the j set will be higher than the maximum of the k set. As such, the model will then be able to generate a probability distribution for the number of j on-treatment measurements that are likely to exceed the maximum of the k off-treatment measurements. The clinical trial data for each individual subject or patient P can be examined directly for the number of on-treatment measurements that exceed the maximum off-treatment measurement. The distribution of the number of j measurements that exceed the maximum k measurement for individuals in the treated group may then be compared with the similar distribution for the placebo-treated or other comparator group.

Differences in the distribution should then be present for the higher numbers of j measurements that exceed the maximum k measurement. These differences allow a suitable criterion to be established for the minimum number of j values exceeding the maximum k value that represents a high likelihood of a treatment response, based on a clear separation of probability in the upper part of the range. A working criterion would be that a treatment response is likely where a majority of the j measurements exceed the maximum k measurement, under the condition that j and k are closely matched small integers (plus or minus one). The probability that the majority of j values lie above the range of k values should be low based on random variability.

Similarly, the clinical trial data may also be compared to the probability distribution from the computer model to check that the probability distribution of the comparator data is similar to the random number model and that there is not a profound deviation from the predictions of the model that would indicate a treatment-period related effect that was independent of treatment.

Once the criterion for response can be established by comparison of the treated and comparator distributions, then in subsequent studies this criterion can be used to identify the numbers of people who appear to respond to treatment in the actively treated and comparator or placebo-treated groups and the significance of differences in response rate can be determined by straightforward statistical testing of those frequency. When configured as such, the characteristics of the response to treatment of the responder group can also then be examined, undiluted by the non-responder population. Neverthless, as can be appreciated, the above descriptions regarding such particulars like the specific comparators employed, the number and type of tests employed, the number or patients, the number of off- and on-treatments may all be modified to suit the particular needs of the clinician and to the specific affliction and/or drug being examined.

Thus, as seen in one exemplary embodiment of the invention, the broadest aspect of the invention may be detailed as comprising a method, a method instantiated or executed on an electronic apparatus such as a computer, and/or a computer readable medium executing the following steps of: identifying a plurality of records relating to patients in a clinical database, said records comprising measurements for patients relating to tests administered during an off-treatment period and an on-treatment period; identifying at least one test in said plurality of records relating to measurements of each individual during an off-treatment period; identifying at least one test in said plurality of records relating to measurements of each individual during an on-treatment period; identifying a baseline measurement of each individual during said off-treatment period; performing a statistical distribution on said plurality of records to identify likelihood of said on-treatment and said off-treatment measurements exceeding said baseline so as to compare said measurements with said baseline; and selecting one or more individuals (“responders”), wherein the selected individuals exhibit an improved performance during a majority of the tests administered during the on-treatment period as compared to a best (e.g., fastest, strongest, etc.) response the test administered to the off-treatment period. However, as can be appreciated, the invention may take the form of a computer readable medium for executing the above detailed steps, or alternatively, may comprise a computer based system for selecting individuals based on responsiveness to a treatment, comprising:

a memory module for storing patient measurements, and for storing at least a first set of instructions relating to the inputting and analyzing of said patient measurements, and a second set of instructions for outputting responder information from said patient measurements; a central processing unit for executing said first and second set of instructions; and an output module for outputting said responder information.

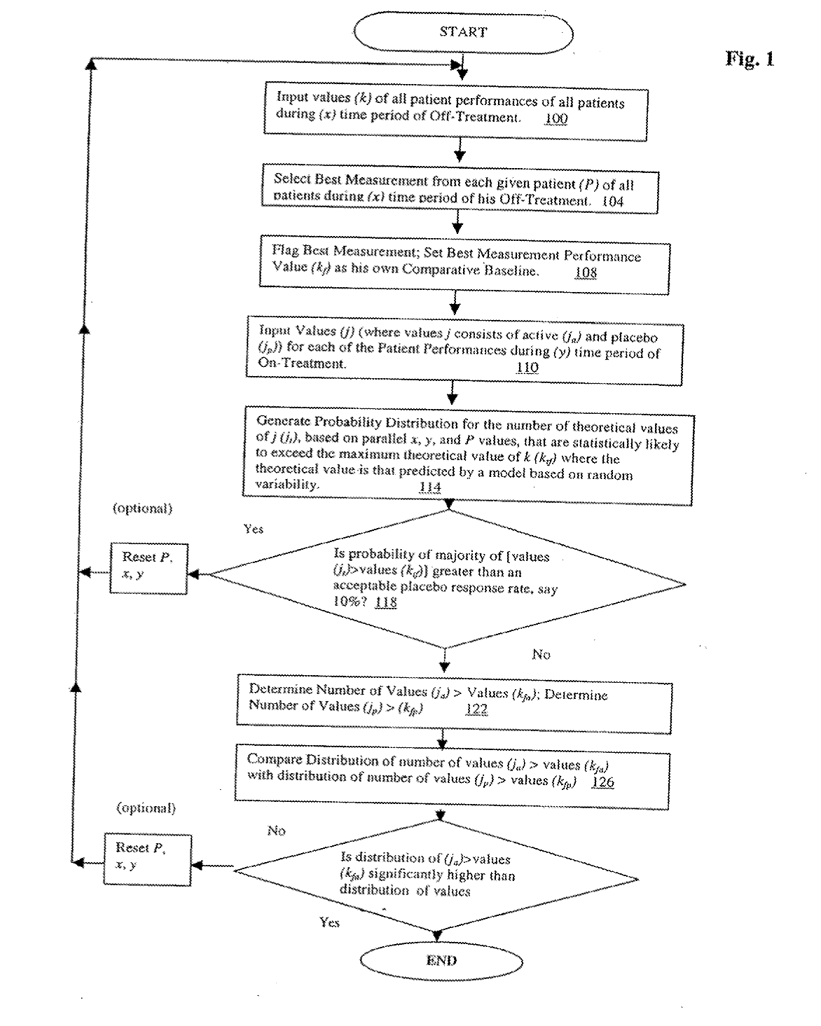

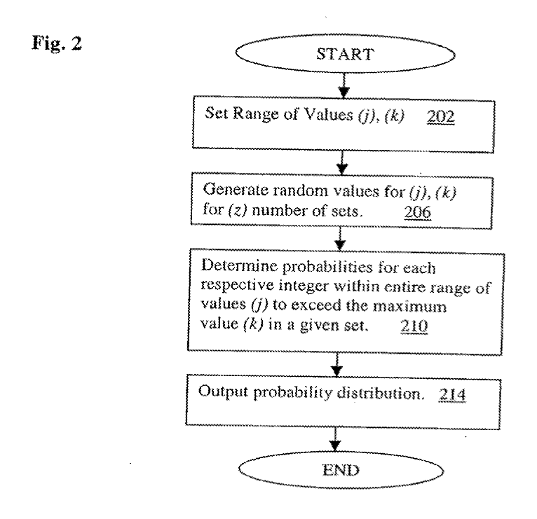

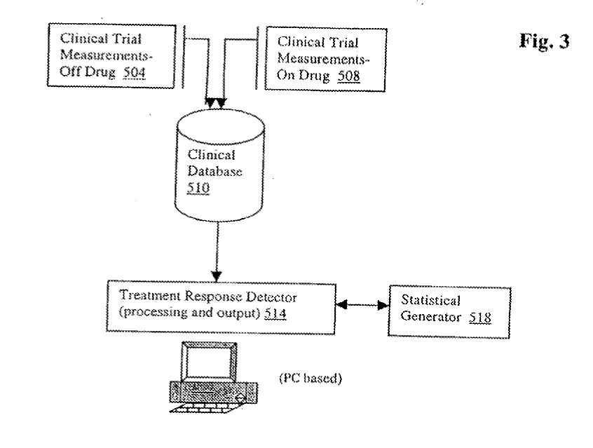

Accordingly, as seen in FIGS. 1, 2, and 3, is art exemplary depiction of the inventive process in: a generalized flow diagram (FIG. 1)(showing steps 100 through 130, with optional resets for re-designing or re-conducting the process so as to reset undesirable results); a generalized flow diagram on one approach to generating a specialized, unique statistical distribution (e.g., step 114 of FIG. 1) used within the overall process (e.g., steps 100-130) in FIG. 1; and an exemplary hardware (apparatus) configuration (FIG. 3), upon which the exemplary flow processes in FIGS. 1 and 2 are executed by the inventive software. The inventive software for executing the above described processes, and for analyzing inputted data, outputs useful information such as responder data. The inventive software and process may be embodied in computer any manner of readable code, and may be contained on any computer readable medium, such as a hard drive (whether PC based, or remote server), disk, CD, etc. When configured as such, the measurements of the patients P are formatted as signals that may be received by the apparatus of FIG. 3 so that the inventive process and software may be transformed into useful outputs for a user. This outputted information may be received by the apparatus in order to be processed and analyzed by the inventive process for use by a user who may receive the outputted signals that have been formed by the steps described herein. As such, the technical effect is such that when the signals are processed in accordance with the above, the tangible, useful result is that clinical trials may be better planned and/or analyzed by researchers who may identify responders to a given treatment for an affliction of almost any nature in ways that were not available heretofore. In any case, in order to achieve this desirable result, it is to be emphasized that the included figures are merely illustrative, and may be reconfigured or revised in many different ways, as one skilled in the art may appreciate.

In a specific exemplary application of one embodiment of the present invention, a method of analyzing the treatment of an illustrative affliction, such as multiple sclerosis is provided. In such an example, the goal might be to employ the general inventive process and software described herein to show the results of a completed clinical study, or otherwise structure a future clinical study that aims to identify responders from a group of patients who receive a given exemplary treatment. In doing so, many indicators may be employed, but in the exemplary illustration indicated in the attached Appendices A, B, C, D and E (each of which is hereby explicitly incorporated by reference in their entireties), such indicators may be such specific measurements as increased walking speed in patients, or increased muscle tone or muscle strength in patients.

Thus, in the given exemplary affliction and clinical treatment depicted in Appendices A, B, C, D, and E, only a proportion of MS pan cuts would typically be expected to have axons of appropriate functional relevance that are susceptible to these drug effects, given the highly variable pathology of the disease. Nevertheless, when the inventive process and software is employed in the manner described herein, and as broadly illustrated in FIGS. 1, 2, and 3, the innovative methodology identifies and characterizes the subset of patients who respond to the exemplary drug fampridine.

To this end, the present invention provides for a method of selecting individuals based on responsiveness to a treatment. In one embodiment, the method comprises identifying a plurality of individuals; administering a test to each individual prior to a treatment period; administering a treatment, including, but not limited to administering a therapeutic agent or drug, to one or more of the individuals during the treatment period; administering the test a plurality of times to each individual during the treatment period; and selecting one or more individuals, wherein the selected individuals exhibit an improved performance during a majority of the tests administered during the treatment period as compared to the test administered prior to the treatment period. In certain embodiments, the method may further comprise administering the test to each individual after the treatment period, wherein the selected individuals further exhibit an improved performance during a majority of the tests administered during the treatment period as compared to the test administered after the treatment period.

It is important to note that this embodiment selects subjects who show a pattern of change that is consistent with a treatment response, but does not define the full characteristics of that response. The criterion itself does not specify the amount of improvement nor does it specify that the improvement must be stable over time. For example, a progressive decline in effect during the course of the study period, even one resulting in speeds slower than the maximum non-treatment value, would not be excluded by the criterion; as a specific example, changes from the maximum non-treatment value of, respectively, +20%, +5%, +1% and −30% during the double blind treatment period would qualify as a response under the criterion, but would actually show a net negative average change for the entire period, poor stability and a negative endpoint. Post-hoc analyses of studies discussed in greater detail below indicate that we may expect responders defined by consistency of effect also to demonstrate increased magnitude and stability of benefit. Thus, as indicated in Appendices A, B, C, D, and E, the existence of a subset of patients who respond consistently to the drug can be supported by quantitative observations in the exemplary clinical studies discussed below.

As further noted in the exemplary application of the inventive process and software on the illustrative clinical trial described in Appendices A, B, D, and E, before treatment, the subjects in these two trials exhibited average walking speeds on the TW25 measure of approximately 2 feet per second (ft/sec). This is a significant deficit, since the expected walking speed for an unaffected individual is 5-6 ft/sec. Subjects in MS-F202 were selected for TW-25 walking time at screening of 8-60, which is equivalent to a range in speed of 0.42-3.1 ft/sec. Variability of functional status is an inherent characteristic of MS, and this can be seen in repeated measurement of walking speed over the course of weeks or months. At any of the three visits during the stable treatment period, 15-20% of placebo-treated subjects showed >20% improvement from baseline walking speed, a threshold chosen as one that is likely to indicate a true change in walking speed over background fluctuations. A larger proportion of the Fampridine-SR treated subjects showed such improvements, but this difference was not statistically significant, given the sample size and placebo response rate.

Given the often large variations in function experienced by people with MS, it is difficult for the subject or a trained observer to separate a treatment-related improvement from a disease-related improvement without the element of consistency over time. Consistency of benefit might therefore be expected to be a more selective measure of true treatment effect than magnitude of change. Based on this rationale, the responses of the individual subjects in the MS-F202 trial were examined for the degree to which their walking speed showed improvement during the double-blind treatment period and returned towards pre-treatment values after they were taken off drug, at follow-up. This subject-by-subject examination yielded a subgroup of subjects whose pattern of walking speed over time appeared to be consistent with a drug response. This led to the analysis illustrated in FIG. 1. This compares the placebo and Fampridine-SR treated groups with respect to the number of visits during the double-blind treatment period in which walking speed on the TW25 was faster than the maximum speed out of all five of the non-treatment visits (four visits prior to randomization and one follow-up visit after the drug treatment period).

The placebo-treated group showed a clear pattern of exponential decline in numbers of subjects with higher numbers of “positive” visits. This is what would be expected from a random process of variability. In contrast, the pattern of response in the Fampridine-SR treated group strongly diverged from this distribution; much larger numbers of Fampridine-SR treated subjects showed three or four visits with higher walking speeds than the maximum speed of all five non-treatment visits and less than half of the expected proportion had no visits with higher speeds. These results indicate that there was a sub-population of subjects in the Fampridine-SR treated group that experienced a consistent increase in walking speed related to treatment.

This analysis suggests that a relatively highly selective criterion for a likely treatment responder would be: a subject with a faster walking speed for at least three (i.e., three or four) of the four visits during the double blind treatment period compared to the maximum value for all five of the non-treatment visits. The four visits before initiation of double-blind treatment provide an initial baseline against which to measure the consistency of response during the four treatment visits. The inclusion of the follow-up visit as an additional component of the comparison was found valuable primarily in excluding those subjects who did not show the expected loss of improvement after coming off the drug. These are likely to be subjects who happened by chance to have improved in their MS symptoms around the time of treatment initiation, but whose improvement did not reverse on drug discontinuation because it was actually unrelated to drug. Thus, incorporating the follow-up visit as part of the criterion may help to exclude false positives, if the TW25 speed remains high at follow-up.

As described in Example 5 in Appendix A, this responder criterion was met by 8.5%, 35.3%, 36.0%, and 38.6% of the subjects in the placebo, 10 mg, 15 mg, and 20 mg treatment groups, respectively, showing a highly significant and consistent difference between placebo and drug treatment groups. Given that there was little difference in responsiveness between the three doses examined, more detailed analyses were performed comparing the pooled Fampridine-SR treated groups against the placebo-treated group. The full results of this analysis for study are described in the following sections. These show that the responder group so identified experienced a >25% average increase in walking speed over the treatment period and that this increase did not diminish across the treatment period. The responder group also showed an increase in Subject Global Impression score and an improvement in score on the MSWS-12. Thus, when utilizing the inventive process and software, it became possible to identify responders experienced clinically meaningful improvements in their MS symptoms, and treatment with fampridine significantly increased the chances of such a response. In doing so, a baseline was established showing comparability among the responder analysis groups, and then analyses were performed on the baseline demographic variables, key neurological characteristics and the relevant efficacy variables at baseline. In general, the responder analysis groups were comparable for all demographic and baseline characteristics variables, with certain exceptions.

Having demonstrated the clinical meaningfulness of consistently improved walking speeds during the double-blind period as a criterion for responsiveness, the question of the magnitude of benefit becomes of interest. The observed differences between the fampridine responders and the placebo group for the functional variables in this study are exactly what we would expect to see in the functional variables in an enrichment study where after a run-in period, only fampridine responders are entered, formed by a washout and randomization to either placebo or fampridine. The fampridine non-responders, although providing no relevant efficacy information, do provide safety information regarding those individuals who are treated with fampridine but show no apparent clinical benefit. As such, responder analyses of these groups were performed.

In one further exemplary embodiment, a method of selecting individuals based on responsiveness to a treatment is derived from executing a range disparity distribution and applying it in a clinical trial setting. In this embodiment, a novel “range disparity” (RD) distribution (RDD) is used to compute the probability that a given number of items (such as patients) in one set fall outside the range, on a give measure, of all the items (patients) in another set. Application of this distribution to evaluation of data from a real clinical trial is described and demonstrates an efficient new form of response analysis. As will be appreciated by those skilled in the art, many additional applications of the range distribution in clinical and other settings may be developed.

The exemplary particulars of the fundamental principle behind a range distribution may be described in the following rudimentary fashion. Suppose that there are three urns; call them X, Y, and Z. Suppose urn Z contains 10 straws of slightly different lengths. A referee selects five straws, places them into urn X and places the remaining five straws into urn Y. What is the probability distribution that a given number of straws in urn Y are longer than the longest straw in urn X?
a The probability is 5 out of 10 for urn X to provide the longest straw. Similarly, there is a 5/10 chance that urn Y will have no straws larger than the largest straw in urn X.
For urn Y to have exactly one straw larger than the largest straw in urn X;
   o urn Y must first have the largest straw (a 5/10 chance);
   the 5 straws in urn X must be the largest among the remaining 9 straws (a 5/9 chance).

So the probability for urn Y to have exactly one straw larger than the largest straw in urn X is ×5/10×5/9.
For urn Y to have exactly two straws larger than the largest straw in urn X, urn Y must first have:
   the largest straw to begin with (a 5/10 chance);
   the second largest straw among the remaining 9 (a 4/9 chance);
   the 5 straws in urn X must be largest among the remaining 8 straws (a 518 chance).

So the probability tar urn Y to have exactly two straws larger than the, largest straw in urn X is ×5/10×4/9×5/8=5/10×5/9×4/8
Continuing this logic, if we let the random variable T represent the number of straws in urn Y that are larger than the largest straw in urn X the we obtain the following distribution:

| t | [P (T = t)] | [P (T = t)] × 100% |
|---|---|---|
| 0 | 5/10 | 50.00% |
| 1 | 5/10 × 5/9 | 27.78% |
| 2 | 5/10 × 5/9 × 4/8 | 13.89% |
| 3 | 5/10 × 5/9 × 4/8 × 3/7 | 5.95% |
| 4 | 5/10 × 5/9 × 4/8 × 3/7 × 2/6 | 1.98% |
| 5 | 5/10 × 5/9 × 4/8 × 3/7 × 2/6 × 1/5 | 0.40% |

Note that the probability of any event (i.e. T ϵ {0, 1, 2, 3, 4, 5}) is 1.

As another example, suppose the urn Z has 8-straws of different length, 5 of which are placed into urn X and 3 into urn Y. What is the probability distribution that a given number of straws in urn Y are longer than the longest straw in urn X? By the equivalent logic described above, we obtain the following distribution:

| t | [P (T = t)] | [P (T = t)] × 100% |
|---|---|---|
| 0 | 5/8 | 62.50% |
| 1 | 5/8 × 3/7 | 26.79% |
| 2 | 5/8 × 3/7 × 2/6 | 8.93% |
| 3 | 5/8 × 3/7 × 2/6 × 1/5 | 1.79% |

Note that the probability of any event (i.e T ϵ {0, 1, 2, 3}) is 1.

Using several combinations of straws in urn X and urn Y, the problem can be generalized for urn X to contain S-straws and urn Y to contain T-straws. This leads to the following definition.

Definition 1: Let N represent the set of positive integers. A random variable Y has the distribution, which we will call the Range Disparity Distribution (RDD) when (for S and TεN and Yε∩0N such that 0≤Y≤T)

$$P ( Y = y ) = \{ \begin{matrix}\frac{S}{S + T} , & y = 0 \\ \frac{S}{S + T} \times {\prod }_{k = 1}^{y} \frac{T - k + 1}{S + T - k} , & otherwise\end{matrix} \qquad ( 1 )$$

This leads to the corresponding cumulative distribution function F(y):

$$F ( y ) = P ( Y \leq y ) = \{ \begin{matrix}0 , & y < 0 \\ \frac{S}{S + T} , & y = 0 \\ {\sum }_{j = 0}^{y} P ( Y = j ) , & otherwise\end{matrix} \qquad ( 2 )$$

While the preceding discussion supplies the probability distribution for the number of cases where items from X exceed the range of the items from Y, the same considerations will cover the opposite case: the number of cases where the items from X tall below the range of the items from Y.

This distribution has numerous potential applications: for example, in a clinical trial where measurements of a particular aspect of disease show essentially random variation with time. In such a case, we may be constrained (for example by clinic visit schedules) to obtain only a small sample of measurements from each patient over the course of a baseline period and a small sample of measurements over a treatment period. The RDD provides a simple and effective way to identify individuals who show an unexpected range-shift in either the positive or negative direction, indicating either a consistent benefit or a consistent worsening, that is temporally associated with the treatment. In addition to making between group comparisons, we can compare the distribution of changes in the placebo group to the expected RDD to identify and measure any temporal changes due to factors such as the placebo effects and natural disease progression or remission.

Consistency of benefit from treatment would be expected to be a more effective measure of response (i.e. of causality) than simply examining the magnitude of change between the average baseline visit and the average treatment visit. This is because a meaningful. consistent benefit may be small in magnitude and a lame random deviation, occurring during any individual measurement, can have a substantial but ultimately meaningless effect on the average value across a small number of sample measurements.

Example 1—Theoretical basis: Assuming a clinical trial such that for each patient there are S off-drug measurements of a particular affected function and T on-drug measurements. Let Y represent the number of on-drug measurements that are better (e.g. more normal) than the best off-drug measurement. Assume Y follows the range disparity (RD) distribution. For example, if there are S=5 off-drug visits and T=5 on-drug visits then the probability distribution of Y, is:

| y | [P (Y = y)] × 100% |
|---|---|
| 0 | 50.00% |
| 1 | 27.78% |
| 2 | 13.89% |
| 3 | 5.95% |
| 4 | 1.98% |
| 5 | 0.40% |

This distribution implies that, if the active treatment has no effect we would expect the proportion of patients who experience a consistent improvement, reflected by 4 or 5 on-drug measurement better than the best off-drug measurement, to be about 2.5%. The null hypothesis that the groups are equal with regard to the proportion of subjects with consistent improvement can be tested using a standard test such as Fisher's exact test, a chi-square test, or for stratified samples (e.g., by study center) the Cochran-Mantel-Haenszel test. Significant departures from this expected frequency in the active treatment group, but not the placebo group, would lead us to conclude that the treatment and placebo groups are different. Significant differences between all three distributions (active treatment, placebo, and expected RD), would indicate a treatment effect superimposed on a temporal change due to other factors.

Hence, the identification of a consistent response as represented by 4 or 5 of the on-drug measurements as better than the best off-drug measurement provides a particularly clear criterion for a responder analysis. A traditional responder analysis would establish an arbitrary level of average. change (e.g. 10%, 20%) above which a trial subject would qualify as a responder. Generally, there is no clear clinical or statistical justification for such a criterion and no a priori method for its estimation. On the other hand, a criterion of consistency based on the RDD can be clinically meaningful (being based on consist relationship to treatment over time), statistically appropriate (based on a threshold of statistical probability, here approximately 2.5% for a one-sided criterion.) and it can be calculated a priori, given the trial design.

Below is a brief outline of a general approach to determine appropriate parameters for a responder criterion based on the concept of consistency across measurements. A general approach to determine an appropriate response criterion for a clinical trial might be derived as follows:

Let,

Xᵢₛ (i=1, 2, . . . , I and s=1, 2, . . . S) represent the s^{th} off-drug measurement for patient i.

Yᵢₜ (i=1, 2, . . . , I and t=1, 2, . . . T) represent the t^{th} on-drug measurement for patient i.

### Assumptions:

Each X and Y measurement addresses the same outcome variable: Z (we use X and Y to differentiate measurements during different time-periods: off-drug and on-drug).
Initially assume no treatment effect and that there is no longitudinal effect on the outcome measure. That is to say that over time, there is at best, negligible within-patient correlation. If such an effect exists, it will become apparent in the analysis itself.
   Set up a consistency criterion C which is a relation (p) between the off-drug measurements and each on-drug measurement such that:

$${C}_{it} = \{ \begin{matrix}1 & if f ( {X}_{is} ) ρ {Y}_{it} \\ 0 & otherwise\end{matrix}$$

$${C}_{i} = {\sum }_{i = 1}^{T} {C}_{it} .$$

and compute the number of on-drug visits that fulfill the criterion choose a value λ≤T such that a responder criterion is defined as:

$${C}_{iR} = \{ \begin{matrix}1 & if {C}_{i} \geq λ \\ 0 & otherwise\end{matrix}$$

For clinical trials, a good rule of thumb is to chooses λ such that the theoretical responder rate is no larger than 5%, i.e.:

0≤[P(C_{iR})=1]≤0.05

Example 2—Practical experience: The following is based on data from a clinical trial that examined the effects of a novel treatment in improving walking speed in patients diagnosed with a chronic disease and was designed with 5 off-drug and 4 on-drug assessments of walking speed. Subjects were randomized to receive active drug or placebo in a 3:1 ratio, For a given patient, if we let Y represent the number of on-drug measured walking speeds that are faster than the fastest off-drug walking speed and assume Y follows the RDD we have:

TABLE 1
Table showing the theoretical distribution of on-drug visits with faster
walking speeds than the fastest off-drug walking speed using the RDD.

| y | [P (Y = y)] × 100% |
|---|---|
| 0 | 55.56% |
| 1 | 27.78% |
| 2 | 11.90% |
| 3 | 3.97% |
| 4 | 0.79% |

Applying the general approach to determine an appropriate response criterion, response to treatment was defined as a faster walking speed in at least 3 of the 4 on-drug visits compared to the fastest speed measured during the 5 off-drug visits. There were 205 intent-to-treat patients included in the primary efficacy analysis (47 placebo and 158 active treatment). Table 2 below summarizes the key study result.

TABLE 2
Table showing the percentage of responders, selected for
consistent improvement in walking speed in the placebo-treated
and active-drug treated groups. P-value calculated from the
Cochran-Mantel-Haenszel test, controlling for study center.

| | Placebo (N = 47) | Drug (N = 158) |
|---|---|---|
| Percentage of Responders | 8.5% | 36.7% |
| p-value | <0.001 | |

As cart be seen, the placebo responder rate (8.5%) was very close to the theoretical responder rate of about 5%. Indeed, when we examine the frequency distribution for the placebo-treated group in Graph A, below, we see that the observed distribution of better on-drug measurements was similar to that expected from the RDD, thereby suggesting negligible temporal or placebo effects in this trial. On the other hand, the distribution of measurements in the actively treated group was significantly different from both the placebo and the theoretical distributions. In particular, there were large differences in the proportion of subjects showing no measurements faster than the fastest off-drug measurement and showing 3 or 4 faster visits. This indicates that active treatment but not placebo treatment is associated with more consistent improvement than would be expected from the RDD, and that our selection of the response criterion based on statistical probability is reasonable in practice.

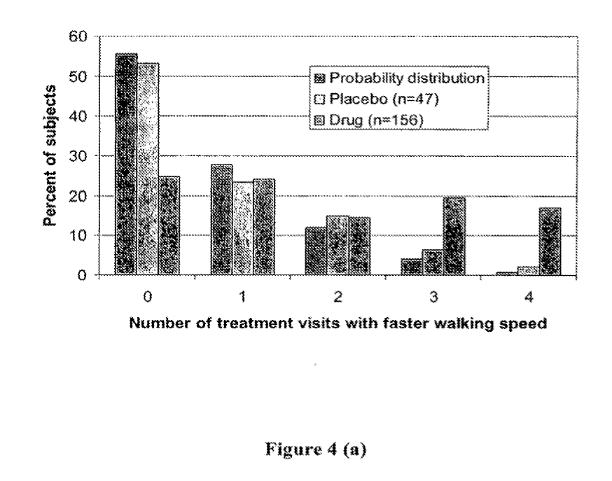

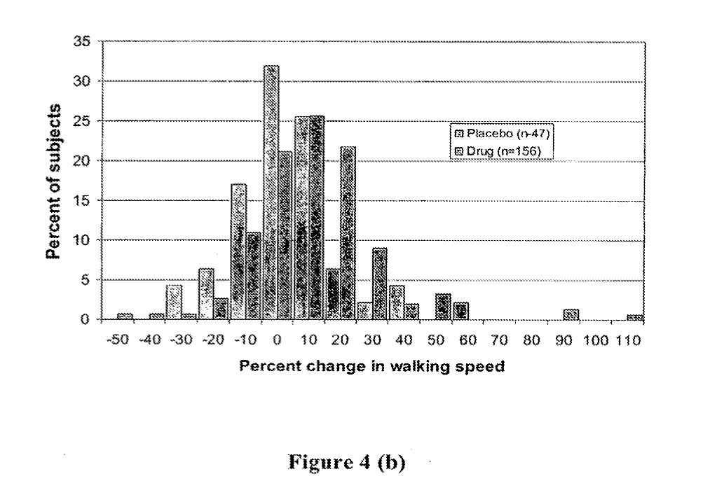

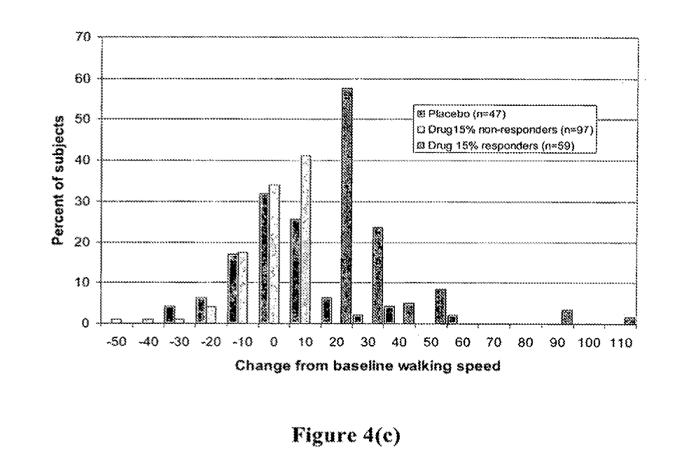

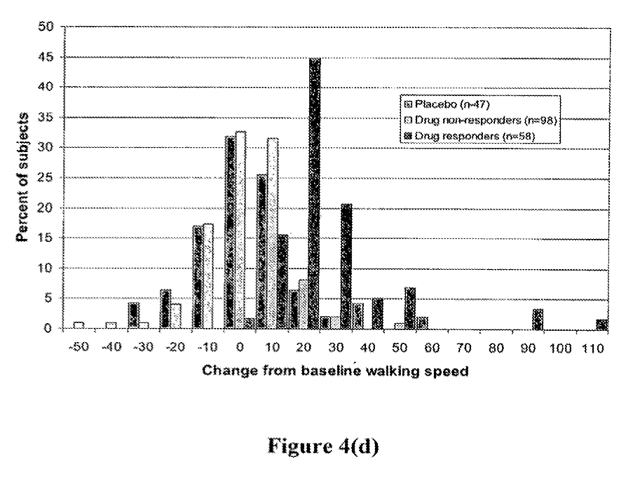

FIG. 4 (a): Histogram to show the proportion of subjects in the two treatment groups who experienced a given number of walking assessments during treatment that were Raster than the fastest speed measured during the off-treatment period. These distributions are compared to the expected RD probability distribution.

The utility of this form of response analysis is shown both by the differentiation of treatment and control groups and by the ability to show that, in the absence of active treatment, there was no significant independent shift in the placebo group that would indicate treatment-independent changes related to time or to placebo-effects.

The application of this criterion for “consistent response analysis” allows very efficient sampling. This is shown by comparing three forms of analyzing the data from this study in FIGS. 4 (b)-(d). Simply by examining the distribution of mean changes between baseline and treatment periods in a traditional quantitative comparison (in FIG. 4(c)) we see a trend for improvement in the drug-treated group, and we would expect such a difference to be resolved with statistical significance in a much larger study). However, in this study the difference was not significant by ANOVA. A traditional response analysis, setting a threshold of 15% in (FIG. 4 (c)) shows again a clear favorable trend, with many more “responders” in the active group, but this is not statistically significant with the sample size available (using the Cochran-Mantel Haenszel test). Similar results are achieved with other arbitrary response thresholds, e.g. 10% or 20%. Applying the “consistent response” criterion, we see a highly statistically significant response with the available sample size (FIG. 4(d), Table 2). Not only is the overall analysis rendered more sensitive by the consistent response criterion, but this approach, unlike the other two, also allows us: a) to show that the placebo response rate is not significantly different from that expected by random variability (FIG. 4 (a), Table 2); and b) to know before the trial that the expected false-positive rate for the criterion is approximately 5% (Table 1) based on the RD probability distribution.

FIG. 4(b): Distribution of changes in average walking speed between the baseline, pre-treatment period and the double-blind treatment period for patients randomized to either placebo treatment or active treatment. Although there appears to be a difference in mean changes between treatment groups, this was not statistically significant based on ANOVA.

FIG. 4 (c): Applying a traditional responder analysis to the data in FIG. 4 (b), with a threshold for “response” set at >15% improvement. This shows that the distribution of changes among non-responders in the drug-treated group is very different from that of the placebo group, indicating the presence of significant numbers of false negatives among non-responders and false positives among responders. A total of 7 placebo-treated patients registered as responders, for a 14.8% false-positive rate in that group. The response rate in the drug-treated group was 44.2%. The drug-placebo response differential was therefore 29.4%, but this was not statistically significant. Clearly, the threshold chosen for the response definition will affect the numbers of responders in both treatment groups but would not change the arbitrary nature of the division between responders and non-responders.

FIG. 4(d): Applying the consistent (repeated measures) response analysis to the same data from FIG. 4 (b) selects out a non-responder population that is close to the placebo-treated group in its distribution. Only 4 subjects in the placebo group registered as responders, for an 8.5% false positive rate. The response rate in the drug-treated group was 37.2%. The drug-placebo response differential was therefore 28.7%, similar to the 29.3% seen with the traditional responder analysis approach shown in FIG. 4 (b). However, in this case there was a lower frequency of false positives in the placebo group, and the difference between treatment group response rates was statistically significant (p<0.001, Table 1).

Example 3: The application of this distribution is particularly powerful in the context of a repeated measures response analysis of the kind provided by Example 2. However, it may be useful in various simpler situations, for example, in a case of industrial product sampling. There might be a suspicion that plant A is producing items with a breaking strength that is lower than those from plant B. For destructive testing of items from the two plants we would likely want to minimize the sample size. If we sampled 5 out of 100 items from the next production run at each plant and determine that the breaking strength of more than 2 of these items from plant A falls below the range of the 5 tested from plant B we would have support for the suspicion regarding a difference between the plants. More specifically, we would know that there is less than a 2.5% chance, on the basis of random variability (Example 1), that 4 or 5 of the samples from A would fall below the failure range for those from plant B.

The range disparity distribution, therefore describes the expected behavior of two small samples from a common population. Specifically, it defines the probability that any given number of values in one sample from that population will fall outside the range of values in the other sample, in either the positive or negative direction. This distribution can be applied to novel forms of small-sample statistical analysis. An example of application to a repeated measures response analysis in a clinical trial is described. The definition of a consistent response, based on the sample range disparity distribution, improves the sensitivity as well as the statistical and clinical meaningfulness of such an analysis.

Example 4: In addition, the method, system and software of the present invention was utilized in the testing of Fampridine-SR on walking in people with multiple sclerosis (MS) during a Phase 3 trial, the results of which were announced on Sep. 25, 2006. In particular, this Phase 3 clinical trial of Fampridine-SR on walking in people with multiple sclerosis (MS) was a confirmation of the pertinence of the inventive approach. In utilizing the method, system and software of the present invention, statistical significance was achieved on all three efficacy criteria defined in the Special Protocol Assessment (SPA) by the Food and Drug Administration (FDA). As a result of utilizing the inventive techniques, a significantly greater proportion of people taking Fampridine-SR. had a consistent improvement in walking speed, the study's primary outcome, compared to people taking placebo (34.8 percent vs. 8.3 percent) as measured by the Timed 25-Foot Walk (p less than 0.001). In addition, the effect was maintained in this study throughout the 14-week treatment period (p loss than 0.001) and there was a statistically significant improvement in the 12-Item MS Walking Scale (MSWS-12) for walking responders vs. non-responders (p less than 0.001). The average increase in walking speed over the treatment period compared to baseline was 25.2 percent for the drug-responder group vs. 4.7 percent for the placebo group. Increased response rate on the Timed 25-Foot Walk was seen across all four major types of MS. In addition, statistically significant increases in leg strength were seen in both the Fampridine-SR Timed Walk responders (p less than 0.001) and the Fampridine-SR Timed Walk non-responders (p=0.046) compared to placebo.

Although the present invention has been described in considerable detail with reference to certain preferred embodiments thereof, other versions are possible. Therefore the spirit and scope of the appended claims should not be limited to the description and the preferred versions contain within this specification.

We claim:

1. A method for selecting individuals based on responsiveness to a treatment, the method comprising the following steps:
identifying a plurality of records relating to patients in a clinical database, said records comprising measurements for patients relating to tests administered during an off-treatment period and an on-treatment period;
identifying at least one test in said plurality of records relating to measurements of each individual during an off-treatment period;
identifying at least one test in said plurality of records relating to measurements of each individual during an on-treatment period;
identifying a baseline measurement of each individual during said off-treatment period;
performing a statistical distribution on said plurality of records to identify likelihood of said on-treatment and said off-treatment measurements exceeding said baseline so as to compare said measurements with said baseline; and
selecting one or more individuals, wherein the selected individuals exhibit an improved performance during a majority of the tests administered during the on-treatment period as compared to a best response to said at least one test administered to the off-treatment period.

2. A computer program product, for use with a computer system, for selecting individuals based on responsiveness to a treatment, the computer program product comprising:
a computer readable medium containing thereon instructions operative to control the operation of a computer system to perform the steps of:
identifying a plurality of records relating to patients in a clinical database, said records comprising measurements for patients relating to tests administered during an off-treatment period and an on-treatment period;
identifying at least one test in said plurality of records relating to measurements of each individual during an off-treatment period;
identifying at least one test in said plurality of records relating to measurements of each individual during an on-treatment period;
identifying a baseline measurement of each individual during said off-treatment period;
performing a statistical distribution on said plurality of records to identify likelihood of said on-treatment and said off-treatment measurements exceeding said baseline so as to compare said measurements with said baseline; and
selecting one or more individuals, wherein the selected individuals exhibit an improved performance during a majority of the tests administered during the on-treatment period as compared to a best response to said at least one test administered to the off-treatment period.

3. A computer based system for selecting individuals based on responsiveness to a treatment said system comprising:
a memory module for storing patient measurements, and for storing at least a first set of instructions relating to the inputting and analyzing of said patient measurements, and a second set of instructions for outputting responder information from said patient measurements;
a central processing unit for executing said first and second set of instructions, and for outputting the responder information resulting from said executing of said first and second instructions, said central processing unit being connected to said memory module, and in operative control of said memory module; and
an output module connected to said central processing unit for displaying said responder information.
